(19) United States
(12) Patent Application Publication
(10) Pub. No.: US 2008/0281190 A1
(43) Pub. Date: Nov. 13, 2008

(54) SURGICAL PROCEDURES

(75) Inventors: Robert J. Petcavich, The Woodlands, TX (US); Murray Reicher, Rancho Santa Fe, CA (US)

Correspondence Address: GREER, BURNS & CRAIN, 300 S WACKER DR, 25TH FLOOR, CHICAGO, IL, 60606, US

(73) Assignee: Health Beacons, Inc., Kirkland, WA (US)

(21) Appl. No.: 12/176,654

(22) Filed: Jul. 21, 2008

Related U.S. Application Data
(63) Continuation-in-part of application No. PCT/US2007/002313, filed on Jan. 25, 2007.
(60) Provisional application No. 60/761,851, filed on Jan. 25, 2006.

(51) Int. Cl. A61B 5/05; A61B 19/00

(52) U.S. Cl. 600/424; 600/427

(57) ABSTRACT

A method of locating and identifying, during surgery, target regions within a body intended for excision of suspect tissue and/or removal of diseased organs or foreign objects, resides in implantation within the body proximate the suspect tissue or object prior to surgery of one or more passive integrated transponder tags and, at the time of and/or during surgery, scanning of the body with a radio frequency scanner or reader that activates the tag or tags and provides the surgeon with one or more signals indicative of the approximate location and unique identification of each of the tags, thereby to aid the surgeon in performance of the surgery. Verification of the success of the surgical procedure is obtained following surgery by scanning the site for absence of the tag or tags and/or by scanning the excised tissue for presence of the tag or tags.

### CROSS-REFERENCE TO RELATED APPLICATIONS

This application is a continuation-in-part application filed under 35 U.S.C. §111(a), of International Application PCT/US2007/002313, filed Jan. 25, 2007, and claims the benefit of U.S. Provisional Application No. 60/761,851, filed Jan. 25, 2006, entitled “Method of Locating Tissue or Foreign Objects Using Implantable Removable Transponders”.

### FIELD OF THE INVENTION

This invention relates to surgery performed on the bodies of humans and animals for removal of suspect tissue, such as cancer, malignancies, tumors and diseased organs, and for removal of foreign objects, such as bullets. The invention is concerned with the identification and the location of suspect tissue for the benefit of an operating surgeon or veterinarian. More particularly, this invention relates to improved methodology to identify the region of surgical interest and the location of tissue that is to be removed by surgery, and subsequent verification of removal of such tissue.

### BACKGROUND OF THE INVENTION

Despite the advances made in technologies such as medical imaging to assist the physician in early stage diagnosis and treatment of patients with possible atypical tissue such as cancer, it is often necessary to sample difficult to reach organs or tissue lesions by biopsy to confirm the presence or absence of abnormalities or disease.

One disease for which biopsy is a critical tool is breast cancer. This affliction is responsible for 18% of all cancer deaths in women and is the leading cause of death among women aged 40 to 55.

In the detection and treatment of breast cancer, there are two general classes of biopsy: the minimally invasive percutaneous biopsy and the more invasive surgical, or “open”, biopsy.

Percutaneous biopsies include the use of fine needles or larger diameter core needles. They may be used on palpable lesions or under stereotactic x-ray, ultrasonic, or other guidance techniques for nonpalpable lesions and microcalcifications (which are often precursors to metastatic cell growth). In the fine needle biopsy, a physician inserts a small needle directly into the lesion and obtains a few cells with a syringe. Not only does this technique require multiple samples, but each sample is difficult for the cytologist to analyze as the specimen cells are isolated outside the context of healthy surrounding tissue.

Larger samples may be removed via core biopsy. This class of procedures is typically performed under x-ray, ultrasound, or MRI guidance in which a needle is inserted into the tissue to obtain a core that is removed via vacuum aspiration, etc. Typically four to five samples are taken from the body. Examples of such biopsy methods include the MAMMOTOME vacuum aspiration system by Johnson & Johnson of New Brunswick, N.J., the ABBI system by United States Surgical Corporation, Norwalk, Conn., and the SITESELECT system by Imagyn, Inc. of Irvine, Calif.

Open biopsies are advisable when suspicious lumps should be removed in their entirety or when core needle biopsies do not render sufficient information about the nature of the lesion. One such type of open biopsy is facilitated by a pre-operative wire localization procedure. The wire localization procedure may be performed with MRI or ultrasound image guidance, but is most often performed with mammographic image guidance, using either conventional film-based mammography, stereotactic mammography imaging, or digital mammographic imaging.

After multiple mammographic images are taken of the breast, the images are analyzed to determine the location of the suspect lesion. Next, after a local anesthetic is administered, and with the aid of an ultrasound, mammographic or other imaging system, a radiologist inserts a small needle into the breast and passes the needle to the suspect tissue. The radiologist then passes a wire with a hook on its end through the needle and positions the hook so that the end of the wire is distal to the suspect tissue. One or more final images are taken of the lesion with the accompanying wire in place, and the radiologist typically then marks the film or digital images to help indicate to the surgeon the relative position of the wire and target tissue requiring removal. The wire is left in the tissue and the patient is taken to the operating room, where the suspect tissue is removed by a surgeon. The removed tissue is then typically sent from the operating room to a radiologist to determine, via x-ray examination, if the tissue contains the x-ray indicators and if the size and borders are adequate to confirm the removal of all suspicious tissue.

Examples of such wire markers are known in the art. See, e.g., the following patents, each of which is incorporated herein by reference: U.S. Pat. No. 5,158,084 to Ghiatas, U.S. Pat. No. 5,409,004 to Sloan, U.S. Pat. No. 5,059,197 to Urie et al., U.S. Pat. No. 5,197,482 to Rank, U.S. Pat. No. 5,221,269 to Miller et al., and U.S. Pat. No. 4,592,356 to Gutierrez. Other similar devices are described in U.S. Pat. No. 5,989,265 to Bouquet De La Joliniere et al. and U.S. Pat. No. 5,709,697 to Ratcliff et al., each incorporated herein by reference.

Despite the advantages of wire localization techniques to locate the suspect tissue for the surgeon, they have a number of limitations.

A wire may move during the post-placement mammographic imaging or transfer of the patient. If a wire is inaccurately placed, it frequently cannot be removed except by surgical excision. Therefore, if a wire is not accurately placed at first, placement of one or more additional wires are required.

Because the distal tip of the wire might have been placed anywhere from the very center of the lesion to quite some distance away from the lesion, the surgeon must guide a scalpel along the wire and rely upon the skill of the radiologist and the marked x-ray or other medical image in the excision procedure. Even if the wire has been properly placed in the lesion and the x-ray film clearly shows the lesion boundary or margin, the surgeon often cannot see the tip of the wire (given the surrounding tissue) so she must remove a larger portion of tissue than is necessary to ensure proper excision.

If the lesion is not found adjacent to the wire, the surgeon may cut or remove non-afflicted tissue without removing the lesion. Also, if the tip of the wire penetrates the lesion, the surgeon may sever the lesion in cutting through the tissue along the wire. In the latter case, a re-excision may be necessary to remove the entire lesion.

Finally, post-excision re-imaging is almost always performed prior to closing the surgical field to ensure that the targeted tissue volume containing the suspect lesion is removed. This requires radiological imaging in the operating room or transport of a specimen outside the operating room to an imaging facility or suite to radiographically confirm excision of the targeted tissue.

When marking lesions in the breast, two paddles are typically used to compress and stabilize the breast for placement of the wire. Upon release of the breast from compression, the wire marker can dislodge or migrate to another position away from the suspect tissue. It may also migrate while the patient awaits surgery. In addition, the fact that the breast is in an uncompressed state during the excision procedure renders a different view of the lesion with respect to the healthy tissue.

Various tissue localization systems have been developed to minimize inadvertent migration of the wire by configuring the wire with a bend or hook, such as Ghiatas et al., discussed above, U.S. Pat. No. 5,011,473 to Gatturna, and the MAMMALOK needle/wire localizer sold by Mitek Surgical Products, Inc., Dedham, Mass. Even if a wire does not migrate after placement, the surgeon generally must follow the wire, which is rarely the most cosmetically desirable path to the lesion (such as a circumareolar approach).

Because the distal tip of the wire is often placed in the center of the suspect tissue, a problem known as “track seeding” can rarely occur in which cancerous or precancerous cells disturbed by the wire or surgical pathway are distributed to unaffected tissue during the procedure.

Additionally, the use of a localization wire marker presents logistical problems. After placement, the wire protrudes from the body. It is necessary for the patient to proceed with the surgical removal of the lesion immediately after wire placement to minimize the chance of infection, wire breakage or disturbance, etc. Thus, when using a hookwire, the localization procedure must be scheduled immediately prior to surgery, requiring coordination of scheduling between a radiologist and a surgeon, and between an image suite and a surgical suite. When using a wire, the surgeon is generally committed to following the same surgical approach that the radiologists used to place the wire. Sometimes, the optimal skin entry position and angle of the wire that is optimal for imaging placement is not the ideal skin entry position for the surgeon and the cosmetic outcome for the patient.

Metallic tags, such as titanium clips, may similarly be implanted in a patient's body to mark the location of suspect tissue, particularly tumors, for the benefit of an operating surgeon or veterinarian. Through highly beneficial, metallic markers suffer many of the same limitations as hookwires. In particular, metallic markers require radiological imaging in the operating room and/or the transport of specimens outside of the operating room to an imaging suite to radiographically confirm that the marker is in the resected lesion.

What is needed is a tissue, organ and object locating device that may be accurately yet removably placed into a target area or surgical site, i.e., a region of tissue that contains suspect tissues, preferably without penetrating or disturbing that volume of tissue. Such a device should reliably define the location and volume of tissue to be removed without the risk of inadvertent migration of the device. Furthermore, a need remains to improve the interaction between the radiologist and the surgeon, to eliminate the need for post-excision x-rays and re-excision, to reduce the overall time for the procedure, and to allow a surgeon to select the shortest or most cosmetically desirable path to the suspect tissue.

### SUMMARY OF THE INVENTION

It is an object of the present invention to provide tissue locating methodology fulfilling the above-enumerated needs.

Another object of the invention is to provide a method of and select apparatus for enabling a surgeon performing an operation to identify and locate suspect tissue, organs and objects in a body, usually from the exterior of the body, remotely of the suspect tissue or object.

It is in particular an object of the invention to provide improved methodology and select apparatus for identifying and locating suspect tissues, organs and objects in a body utilizing microencapsulation and radio frequency technologies.

More particularly, the invention resides in a method of use of microencapsulated, implantable, passive integrated transponder (PIT) elements or tags, a device, for example a thin needle syringe or deployment device, for implanting one or more of the miniature PIT tags proximate a target organ, tissue or object, and a radio frequency probe, scanner or reader manipulated externally of the body for locating and identifying the implanted tag or tags and thus the location and volume of tissue to be excised by the surgeon.

The locator PIT element is preferably partially or totally radiopaque and adapted to penetrate tissue so that at least a portion of the locator element defines a tissue border along a first path. The tissue border defines a volume of tissue for subsequent excision along the border, and contains a target region that is substantially bounded by or in proximity to the PIT locator element.

This invention involves placing a removable locator element in tissue. This is accomplished by penetrating through tissue at a first site to create a port or a pathway for accessing a targeted tissue volume to be excised, inserting a deployment needle or device into the area of tissue of interest and using the needle or device to deploy and implant the PIT element.

The PIT locator element may be placed under x-ray guidance, stereotactic x-ray guidance, ultrasonic guidance, magnetic resonance imaging guidance, and the like. Target region visibility may be enhanced by, for example, the placement or injection of an echogenic substance, such as collagen, hydrogels, microspheres, or other like biocompatible materials, or by the injection of air or other biocompatible gases or contrast agents.

Second, third and even more locator PIT elements or tags may be advanced through the distal end of the deployment needle or device to penetrate tissue so that at least portions thereof further define the tissue border along second, third and/or more paths. The additional paths may be parallel or non-parallel to the first path and may be angularly displaced with respect thereto at any angle or angles the radiologist desires.

The method includes the step of excising the tissue volume defined by the one or more PIT locator elements. This may be accomplished by surgically accessing the locator element and cutting tissue substantially along the surface of the locator element opposite the surface that is disposed immediately adjacent the tissue volume.

The method also includes the use of an external hand held or stationary probe, scanner or reader that locates the PIT element, imparts electrical power to the PIT element and causes the element to transmit back to the antenna probe a signal of its location and an unique identification number.

At the conclusion of or following surgery, but before closing the surgical field or incision, the radio frequency probe or scanner is again employed to scan one or the other or both of the surgical field, to determine the absence therefrom of the PIT tag or tags, and the excised tissue, to determine the presence therein of the tag or tags, thereby to ensure that the target tissue, organ or object has been successfully excised. This step eliminates or at least minimizes the need for radiological re-imaging in the operating room and/or transporting excised tissue from the operating room to an imaging facility for re-imaging and x-ray analysis and/or for re-excision of tissue.

The invention thus assures more efficient and reliable location and identification of target tissue, more precise and reliable excision of the tissue, significantly less reliance on and/or need for re-imaging and re-excision, and less time consuming and more efficient and practicable surgical procedures then provided by prior practices.

These and other objects and advantages of the invention will become apparent to those of reasonable skill in the art from the following detailed description, as considered in conjunction with the accompanying drawings.

### BRIEF DESCRIPTION OF THE DRAWINGS

FIG. 1 is a side view of a radio frequency responsive implantable passive integrated transponder element or tag useful in practice of the present invention;

FIG. 2 is a side view of an illustrative hypodermic syringe that is representative of devices useful as an implantation or deployment device in practice of the invention; and

FIG. 3 is a plan view of a radio frequency scanner/reader useful for practice of the invention.

### DETAILED DESCRIPTION

The following is a detailed description of an embodiment of the invention presently deemed by the inventors to be the best mode of carrying out their invention.

The invention described herein is appropriate for a range of applications for marking specific volumes of tissue or foreign objects for surgical excision or other purposes. Although the description is largely in the context of marking nonpalpaple lesions in breast tissue for subsequent excision, the invention is not so limited. For instance, the invention may be used to mark tissue in a variety of locations in the body of a human being or an animal, such as the liver, the lungs, muscle tissue, bones, or other tissue or organs where the advantages of the invention may be realized. It may also be used to mark foreign objects in tissue or body cavities, such as a bullet or the like. Accordingly, the invention as described and claimed below is not limited to the marking and removal of lesions in breast tissue.

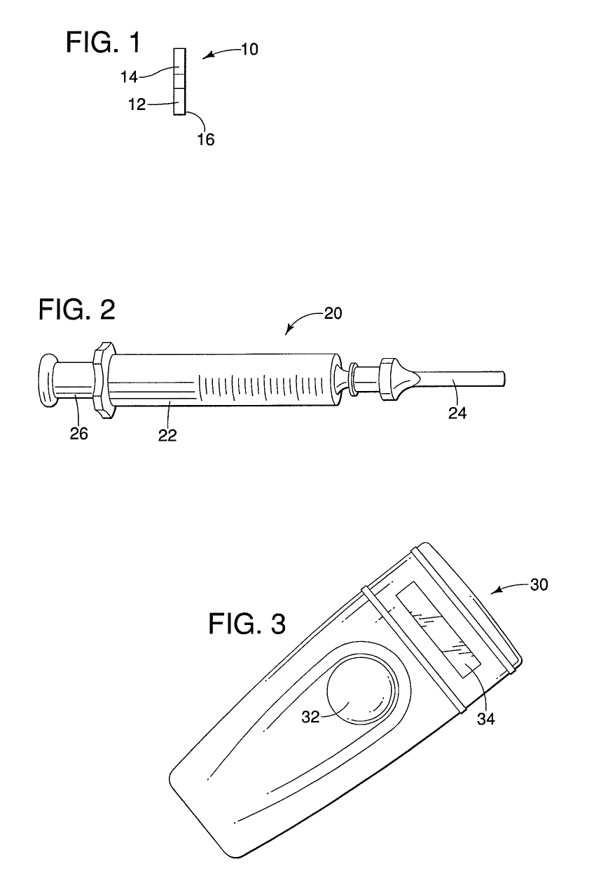

FIG. 1 illustrates an implantable, passive integrated transponder (PIT) element or tag 10 useful in practice of the invention. The PIT element 10 comprises a passive integrated transponder 12 attached to a microchip 14 and encapsulated in an implantable glass shell 16. Passive integrated transponders have no battery so the micro chip remains inactive until energized by radio or other low frequency energy from a scanner or reader, such as the scanner/reader 30 illustrated in FIG. 3.

The micro chip 14 in the tag 10 is pre-recorded with a unique code, such as an alphabetic, numeric or alphanumeric code. The scanner sends a low frequency signal to the transponder and micro chip within the tag providing power needed to interrogate the chip and send its unique code back to the scanner thereby to positively identify the tag or element and provide its approximate location in the body.

The distance from which the PIT tag can be read is called the read range. Many factors contribute to the read range of passive tags including operating frequency, antenna power, tag orientation and interference from other sources. Low frequency tags are detected in milliseconds at close range. PIT elements can be read through materials such as plastic, water and living tissue. In the present invention, it is significant that the PIT elements can be read from a distance of 0.1 to 20 centimeters, with a preferred range of from immediate proximity up to about 7-10 centimeters. A currently useful range is from proximity up to about 7 centimeters.

The frequency used by the PIT element to transmit its location and information is also significant. The present invention utilizes 134.2 KHz as its most preferred transponder frequency so as not to be absorbed by or interfered with by living tissue, body fluids, or water. The range of useful frequencies can be from 1 Hertz to 5 Giga Hertz with 13 Kilo Hertz (kHz) to 150 kHz being preferred.

Implantation or deployment in a body of one or more of the implantable PIT tags 10 is suitably accomplished by use of any of a number of tag deployment needles or devices, for example, a hypodermic syringe, such as the syringe 20 illustratively depicted in FIG. 2. The syringe 20, as is conventional, includes a body 22, a hollow needle 24 for penetrating tissue and a plunger 26 for ejecting a contained substance or object (a PIT tag) through the needle 24 into the body tissue or cavity.

A radiologist, using one or more of the above-referenced imaging techniques, usually determines the region or location of interest, that is, the tissue, volume of tissue, growth, tumor, gland or object to be surgically removed from the body. This location is herein referred to as the “target”, or more fully, the “target tissue, gland or object.” Once the radiologist determines the location of the target in a patient, one or more PIT elements or tags 10 can be implanted in the body at, adjacent, or in proximity to the target by injection via the syringe 20 or other implanter or deployment needle or device. In practice of the invention, the PIT element has a size range of 1 millimeter in diameter to 5 millimeters in diameter, with 2-3 millimeters being preferred, and a length of 2-30 millimeters, with 8-12 millimeters being preferred. In the present invention, a syringe, for example, can be loaded with a PIT element from the point side and into a needle that ranges in size from 1 to 10 centimeters long, with 3-5 centimeters long being preferred, with a needle diameter of from 6 to 20 gauge in opening diameter, with 8 to 12 gauge being preferred. In practice of the invention, each PIT element is pre-loaded into an implanter, gas sterilized, and individually packaged for subsequent use.

A reader or scanner 30 capable of locating and detecting PIT elements or tags 10 implanted in a body is depicted illustratively in FIG. 3. The system employed in practice of the invention is preferably a radio frequency identification system. Radio frequency identification (RFID) uses a signal transmitted between an electronic device such as a PIT element and a reading device such as a scanner or reader or transceiver. RFID technology identifies objects remotely through the use of radio frequencies. In the present invention, RFID is used to locate and identify a PIT element that has been implanted into a patient who will undergo surgery to remove the target tissue, organ or object. The scanner can, with relative ease, detect PIT elements embedded up to 7-10 centimeters deep in a body and give the location and identity of each PIT element to the surgeon without the aid of radiological techniques.

The scanner/reader 30 illustrated is hand held and battery operated, and contains a radio frequency transmitter and receiver (transceiver), an on/off control button 32 and an LCD read-out screen 34 for displaying the unique code pre-recorded in the microchip of each PIT tag.

In practice of the present invention, radiological techniques, such as X-ray, ultrasound and/or magnetic resonance imaging, may be employed to locate targets, that is, suspect tissue, diseased organs, tumors, foreign objects, etc., in a body. A radiologist, using one or more PIT tags and tag implanters, then marks the location by deploying and implanting one or more tags in the body within, adjacent or contiguous to the suspect tissue or organ. Two or more tags may, for example, be implanted to bracket, or to outline the boundaries of, a malignancy, tumor or the like. Once implanted, the tag or tags will maintain their position in the body and will not migrate or be displaced from the location of deployment even though surgery may be delayed for hours, days, or longer.

When the patient is taken to the operating room and prepared for surgery, the scanner control button 32 is depressed and the scanner is passed over the patient's body. A radio frequency signal generated by the scanner activates each PIT element when the scanner is within 7-10 centimeters of the element, and a unique alpha numeric character string is then displayed on the LCD screen 34 that identifies the PIT tag and can be used to localize each tag individually. The surgeon can then use the PIT element or elements as a marker or markers for excising the target tissue, organ or object. Then, when the surgery has been completed, but before closing the surgical field or incision, the scanner can be energized and passed over the excised matter for the presence of PIT signals thereby to determine whether the tag or tags have been excised with the matter, and/or the scanner can be energized and passed over the area where surgery was performed for the absence of PIT signals, thereby to determine whether all of the marked tissue was in fact excised. This feature of the invention may, if desired, be used in conjunction with other locating systems, such as hookwires, to insure thorough excision of target tissue. Thus, the success of a surgical procedure can be determined in the operating room before closing and without need for radiological re-examination and/or surgical re-excision. Specifically, this invention enables real time assessment of the PIT (marker) position within a patient and within a specimen, without the need for re-imaging in the operating room, or transport of a specimen outside of the operating room to radiographically confirm that the marker is in the resected lesion.

The objects and advantages of the invention have thus been shown to be attained in a convenient, practical, economical and facile manner.

While a preferred embodiment of the invention has been herein illustrated and described, it is to be appreciated that various changes, rearrangements and modification may be made therein, and that equivalents thereto may be practiced, without departing from the scope of the invention as defined by the appended claims.

What is claimed is:

1. A method of locating within a body target tissue, organs and objects intended for surgery comprising the steps of
providing at least one implantable transponder,
providing a transponder implantation device,
providing a transponder locating device,
prior to surgery, implanting a transponder within the body proximate the target tissue, organ or object, and
at the time of surgery, scanning the body for the transponder with the transponder locating device.

2. A method as set forth in claim 1 including the step of implanting a plurality of implantable transponders proximate the target tissue, organ or object prior to surgery and, at the time of surgery, scanning the body for the transponders with the transponder locating device.

3. A method as set forth in claim 1 including the step of scanning the body with the transponder locating device following surgery.

4. A method as set forth in claim 1 including the step of scanning surgically excised matter with the transponder locating device following surgery.

5. A method as set forth in claim 1 including the steps of scanning the surgical site and surgically excised matter with the transponder locating device following surgery.

6. A method of locating and identifying within a body target tissue, organs and objects intended for surgery comprising the steps of
providing at least one implantable transponder containing an identifier,
providing a transponder identification reading device,
prior to surgery implanting a transponder within the body proximate the target tissue, organ or object, and
at the time of surgery, locating and identifying the transponder by scanning the body for the transponder with the transponder identification reading device.

7. A method as set forth in claim 6 including the steps of providing a plurality of implantable transponders each containing an identifier, implanting the plurality of implantable transponders within the body proximate the target tissue, organ or object prior to surgery and, at the time of surgery, scanning the body for the transponders with the transponder identification reading device.

8. A method as set forth in claim 6 including the steps of scanning the body and/or and surgically excised matter with the transponder identification reading device following surgery.

9. A method as set forth in claim 6 wherein the transponder comprises a radio frequency responsive passive integrated transponder and the transponder identification reading device comprises a radio frequency transmitter and receiver.

10. A procedure for use in surgically removing target tissue, organs and objects from a body comprising the steps of
prior to surgery, implanting at least one implantable transponder containing an identifier within the body proximate the target tissue, organ or object,
at the time of surgery, locating and identifying the target tissue, organ or object, by scanning the body with transponder locating and identifier reading apparatus, and
following surgery, scanning one or the other or both of the body and surgically excised matter with the transponder locating and identifier reading apparatus.

11. A method for use in surgically removing target tissue, organs and objects from a body comprising the steps of
prior to surgery, implanting at least one implantable transponder containing an identifier within the body proximate the target tissue, organ or object,
at the time of surgery, locating and identifying the target tissue, organ or object, by scanning the body with transponder locating and identifier reading means,
excising the target tissue, organ or object, and
following excision, scanning one or the other or both of the body and the surgically excised matter with the transponder locating and identifier reading means.

12. A method as set forth in claim 11 including the steps of excising the implantable transponder from the body together with the target tissue, organ or object, and scanning the surgically excised matter for presence of the transponder and/or scanning the body for absence of the transponder.

13. A method as set forth in claim 12 wherein the steps recited in claim 12 are completed before closing the surgical field.

14. A method as set forth in claim 11 including the steps of
implanting a plurality of transponders each having an identifier proximate the boundaries of the target tissue, organ or object,
excising tissue from the body along the margins of the transponders distal to the target tissue, organ or object,
scanning the surgically excised matter for presence of the transponders,
and/or scanning the body for absence of the transponders.

15. A method as set forth in claim 14 wherein the steps recited in claim 14 are completed in the operating room prior to closing the surgical field.

16. A method as set forth in claim 11 wherein the target tissue is a tumor.

17. A method as set forth in claim 11 wherein the target tissue is a malignancy in the body.

18. A procedure for use in the surgical removal of target tissue, organs and objects from a body comprising the steps of
providing at least one radio frequency responsive passive integrated transponder containing an identifier,
providing radio frequency transmitting and receiving means for energizing the passive integrated transponder, for reading the identifier in the energized transponder, and for locating the energized transponder,
prior to surgery, implanting the at least one passive integrated transponder in the body proximate the target tissue, organ or object, and
at the time of surgery, scanning the body with said means for remotely energizing the at least one implanted transponder when said means comes into proximity with the transponder, for remotely reading the identifier in the implanted energized transponder, and for remotely determining the location within the body of the implanted energized transponder.

19. A procedure as set forth in claim 18 including the step of energizing the at least one transponder at a radio frequency within the range of from about 13 kHz to about 150 kHz.

20. A procedure as set forth in claim 18 including the step of energizing the at least one transponder for reading by said means at a range of up to about 10 centimeters.

21. A method of scanning, comprising the steps of:
providing one or more targets each identified as a suspect target to be removed from a body;
associating each of said suspect targets with a respective transponder located in the body;
removing one or more of said transponders and its associated suspect target from the body;
scanning the body to determine a presence or absence of said transponders in the body.

22. The method of claim 21, wherein said suspect target is one of a body target tissue, organs, and objects intended for surgery.

23. The method of claim 21, further comprising the steps of:
(a) removing at least one of said transponders from the body when a scan indicates a presence of at least one of said transponders is present in the body;
(b) scanning the body to determine a presence or absence of said transponders in the body; and
(c) repeating steps (a) and (b) until said scanning step determines an absence of said transponders in the body.

24. The method of claim 21, wherein the providing step further comprises the step of locating the one or more suspect targets by performing one of X-ray imaging, ultrasound, and magnetic resonance imaging of the body.

25. The method of claim 24, wherein the associating step comprises implanting a corresponding transponder adjacent to or within each of said located suspect targets.

26. The method of claim 21, further comprising the step of energizing each of said transponders at a radio frequency within a range of from about 13 kHz to about 150 kHz.

27. The method of claim 21, further comprising the step of energizing each of said transponders for reading at a range of up to 10 centimeters.
